(19) United States
(12) Patent Application Publication
(10) Pub. No.: US 2025/0204910 A1
(43) Pub. Date: Jun. 26, 2025

(54) SURGICAL STAPLERS WITH REMOVABLE AND REUSABLE CONTROL ATTACHMENTS

(71) Applicant: CILAG GMBH INTERNATIONAL, ZUG (CH)

(72) Inventors: MATTHEW D. COWPERTHWAIT, CINCINNATI, OH (US); SHANE R. ADAMS, LEBANON, OH (US); ERIC LAFAY, MADEIRA, OH (US)

(21) Appl. No.: 18/395,200

(22) Filed: Dec. 22, 2023

(51) Int. Cl. A61B 17/068; A61B 17/00; A61B 46/10

(52) U.S. Cl. CPC A61B 17/068; A61B 46/10; A61B 2017/00017; A61B 2017/0023; A61B 2017/00464; A61B 2017/00734

(57) ABSTRACT

The present disclosure provides a stapler system. The stapler system includes a disposable surgical stapler and a removable and reusable control attachment. The control attachment includes an energy source in electrical connection with a motor assembly in the disposable surgical stapler. The control attachment includes a controller in electrical communication with the motor assembly and configured to output a motor drive signal to the motor assembly such that the motor assembly drives at least a portion of the firing assembly.

### FIELD OF THE INVENTION

The present disclosure generally describes surgical instruments with a removable and reusable control attachment and, more particularly, to surgical staplers with a removable and reusable control attachment that includes a controller and an energy source that is in in electrical communication with a motor assembly.

### BACKGROUND

In some surgical procedures (e.g., colorectal, bariatric, thoracic, etc.), portions of a patient's digestive tract (e.g., the gastrointestinal tract and/or esophagus, etc.) may be cut and removed to eliminate undesirable tissue or for other reasons. Once the tissue is removed, the remaining portions of the digestive tract may be coupled together in an end-to-end anastomosis. The end-to-end anastomosis may provide a substantially unobstructed flow path from one portion of the digestive tract to the other portion of the digestive tract, without also providing any kind of leaking at the site of the anastomosis.

One example of an instrument that may be used to provide an end-to-end anastomosis is a circular stapler. Some such staplers are operable to clamp down on layers of tissue, cut through the clamped layers of tissue, and drive staples through the clamped layers of tissue to substantially seal the layers of tissue together near the severed ends of the tissue layers, thereby joining the two severed ends of the anatomical lumen together. The circular stapler may be configured to sever the tissue and seal the tissue substantially simultaneously. For instance, the circular stapler may sever excess tissue that is interior to an annular array of staples at an anastomosis, to provide a substantially smooth transition between the anatomical lumen sections that are joined at the anastomosis. Circular staplers may be used in open procedures or in endoscopic procedures. In some instances, a portion of the circular stapler is inserted through a patient's naturally occurring orifice.

As surgical devices such as staplers integrate more advanced electronic features, and reclamation and waste streams become more important, modularity and reusability (or at least partial reusability) of the devices become more critical. These and other problems exist.

### BACKGROUND

The present disclosure provides solutions to the needs mentioned above. One aspect of the present disclosure provides a stapling system. The stapling system includes a disposable surgical stapler. The disposable surgical stapler includes a firing assembly configured to deploy staples from a stapling head assembly. The disposable surgical stapler includes a handle assembly mechanically coupled to the firing assembly. The disposable surgical stapler includes a motor assembly disposed within the handle assembly and configured to drive the firing assembly. The stapling system includes a disposable surgical stapler a removable and reusable control attachment. The control attachment includes an energy source in electrical connection with the motor assembly. The control attachment includes a controller in electrical communication with the motor assembly and configured to output a motor drive signal to the motor assembly such that the motor assembly drives at least a portion of the firing assembly.

In any embodiment described herein, the handle assembly can include a housing cavity, wherein the control attachment is positionable within the housing cavity, and wherein once the control attachment is positioned within the housing cavity, the control attachment is aseptically sealed within the housing cavity.

In any embodiment described herein, the handle assembly can include a cover configured to aseptically seal the energy source and the controller within the housing cavity.

In any embodiment described herein, the controller is in electrical communication with the motor assembly via a hall effect switch.

In any embodiment described herein, the controller is in electrical communication with the motor assembly via an inductive switch.

In any embodiment described herein, the disposable surgical stapler can include a motor activation module. The disposable surgical stapler can include a firing trigger. Activation of the firing trigger actuates a switch of motor activation module to activate the motor assembly. The controller transmits the motor drive signal in response to activation of the motor assembly by the motor activation module.

In any embodiment described herein, the disposable surgical stapler can include a current sensor configured to measure the current drawn by the motor assembly from the energy source.

In any embodiment described herein, the control attachment can include a current sensor configured to measure the current drawn by the motor assembly from the energy source.

In any embodiment described herein, the energy source is a rechargeable battery.

Another aspect of the present disclosure provides method of operating a surgical stapler.

The method includes inserting a removable and reusable control attachment into a first housing cavity of a first disposable surgical stapler. The control attachment can include an energy source and a controller, and the first disposable surgical stapler can include a first motor assembly and a first firing assembly. The method includes actuating a first firing trigger of the first disposable surgical stapler, wherein actuating the first firing trigger causes a first activation signal to be transmitted to the controller and a first motor drive signal to be transmitted from the controller to the first motor assembly such that the first motor assembly drives at least a portion of the first firing assembly. The method includes removing the control attachment from the first housing cavity.

In any embodiment described herein, the method can include recharging the energy source.

In any embodiment described herein, the method inserting the control attachment into a second housing cavity of a second disposable surgical stapler. The second disposable surgical stapler can include a second motor assembly and a second firing assembly. The method can include actuating a second firing trigger of the second disposable surgical stapler. Actuating the second firing trigger causes a second activation signal to be transmitted to the controller and a second motor drive signal to be transmitted from the controller to the second motor assembly such that the second motor assembly drives at least a portion of the second firing assembly and drive a plurality of staples. The method includes removing control attachment from the second housing cavity.

In any embodiment described herein, once the control attachment is positioned within the first housing cavity, the control attachment can be aseptically sealed within the first housing cavity.

In any embodiment described herein, the method can include closing a cover on the first disposable surgical stapler to aseptically seal the control attachment with the first housing cavity.

In any embodiment described herein, the controller can be in electrical communication with the motor assembly via a hall effect switch or via an inductive switch.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of an exemplary circular stapler, according to one aspect of the present disclosure;

FIG. 2 depicts a perspective view of the circular stapler of FIG. 1, with a control attachment removed from a handle assembly and an anvil removed from a stapling head assembly, according to one aspect of the present disclosure;

FIG. 3 depicts a perspective view of the anvil of the circular stapler of FIG. 1, according to one aspect of the present disclosure;

FIG. 4 depicts a perspective view of the stapling head assembly of the circular stapler of FIG. 1, according to one aspect of the present disclosure;

FIG. 5 depicts an exploded perspective view of the stapling head assembly of FIG. 4, according to one aspect of the present disclosure;

FIG. 6 depicts an exploded perspective view of the circular stapler of FIG. 1, with portions of the shaft assembly shown separately from each other, according to one aspect of the present disclosure;

FIG. 7A depicts a cross-sectional side view of the anvil of FIG. 3 positioned within a first section of a digestive tract and the stapling head assembly of FIG. 4 positioned in a second section of the digestive tract, with the anvil separated from the stapling head assembly, according to one aspect of the present disclosure;

FIG. 7B depicts a cross-sectional side view of the anvil of FIG. 3 positioned within the first section of the digestive tract and the stapling head assembly of FIG. 4 positioned in the second section of the digestive tract, with the anvil secured to the stapling head assembly, according to one aspect of the present disclosure;

FIG. 7C depicts a cross-sectional side view of the anvil of FIG. 3 positioned within the first section of the digestive tract and the stapling head assembly of FIG. 4 positioned in the second section of the digestive tract, with the anvil retracted toward the stapling head assembly to thereby clamp tissue between the anvil and the stapling head assembly, according to one aspect of the present disclosure;

FIG. 7D depicts a cross-sectional side view of the anvil of FIG. 3 positioned within the first section of the digestive tract and the stapling head assembly of FIG. 4 positioned in the second section of the digestive tract, with the stapling head assembly actuated to sever and staple the clamped tissue, according to one aspect of the present disclosure;

FIG. 7E depicts a cross-sectional side view of the first and second sections of the digestive tract of FIG. 7A joined together at an end-to-end anastomosis, according to one aspect of the present disclosure;

FIG. 8 is block diagram of an example firing system for a circular stapler such as the surgical circular stapling instrument illustrated in FIG. 1, according to one aspect of the present disclosure; and

FIG. 9 is a flowchart depicting an example method of operating a surgical stapler, according to one aspect of the present disclosure.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

### I. Overview of Exemplary Circular Stapling Surgical Instrument

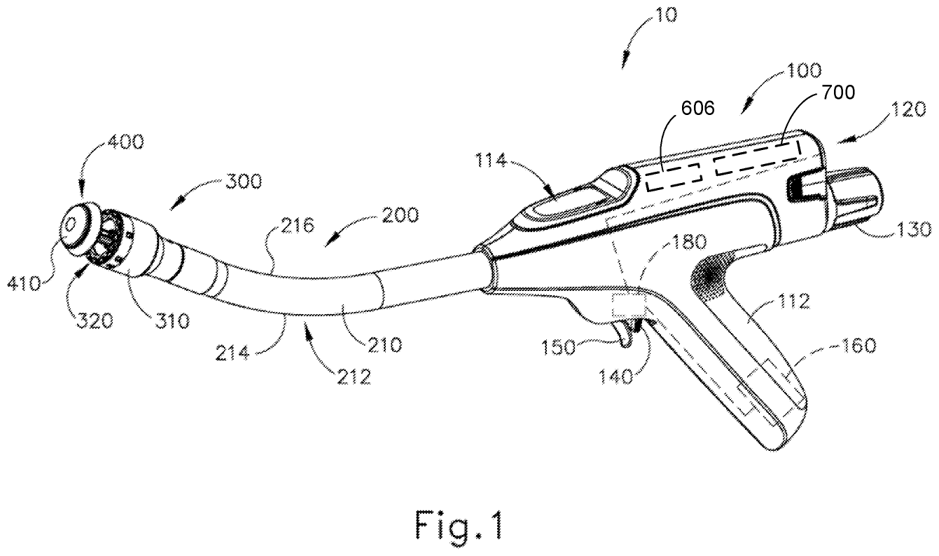

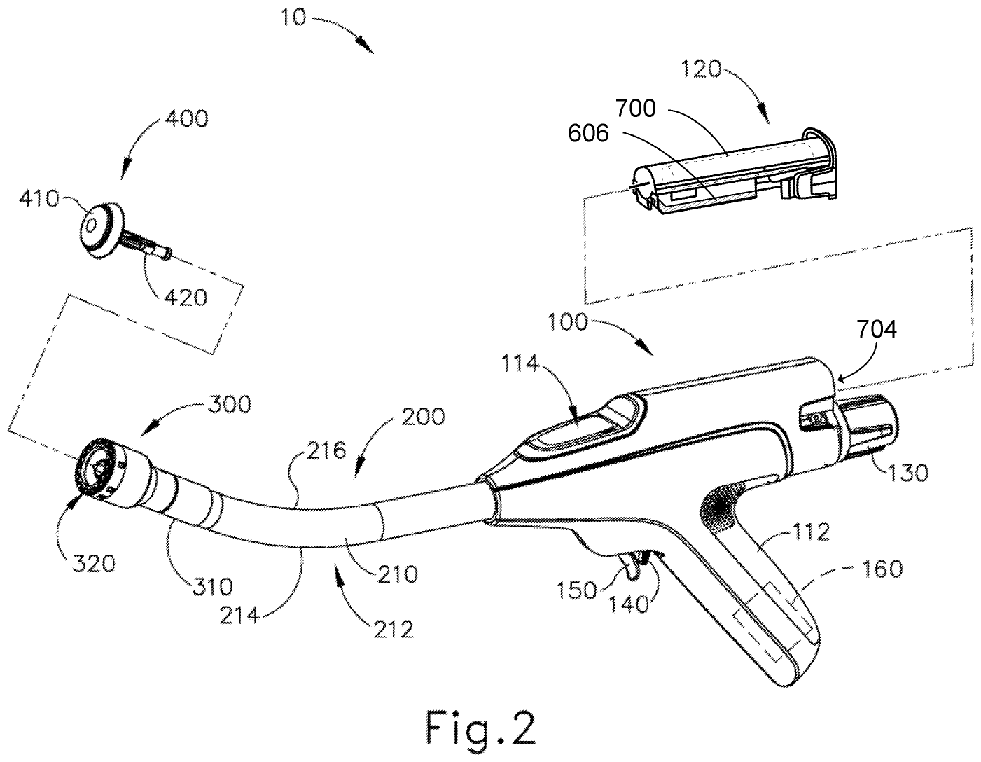

FIGS. 1 and 2 depict an exemplary surgical stapler (10) that may be used to provide an end-to-end anastomosis between two sections of an anatomical lumen such as a portion of a patient's digestive tract. Surgical stapler (10) of this example comprises a handle assembly (100), a shaft assembly (200), a stapling head assembly (300), an anvil (400), and a removable control attachment (120). Each of these components will be described in greater detail below. As will be appreciated, the surgical staplers (10) shown in the figures each show a circular stapler, which is in accordance with a preferred embodiment. That said, the disclosure herein is not so limited, and the features described herein can also apply to other surgical staplers, including for example linear staplers.

### A. Exemplary Tissue Engagement Features of Circular Stapling Instrument

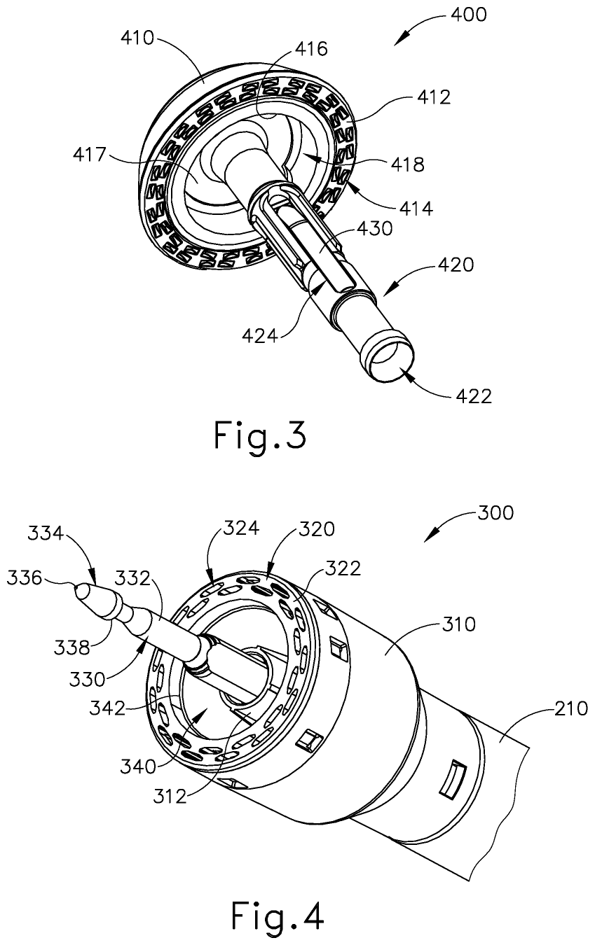

As best seen in FIG. 3, anvil (400) of the present example comprises a head (410) and a shank (420). Head (410) includes a proximal surface (412) that defines a plurality of staple forming pockets (414). Staple forming pockets (414) are arranged in two concentric annular arrays in the present example. Staple forming pockets (414) are configured to deform staples as the staples are driven into staple forming pockets (414) (e.g., deforming a generally “U” shaped staple into a “B” shape as is known in the art). Shank (420) defines a bore or lumen (422) and includes a pair of pivoting latch members (430) positioned in bore (422). Each latch member (430) includes features that allows anvil (400) to be removably secured to a trocar (330) of stapling head assembly (300) as will be described in greater detail below. It should be understood, however, that anvil (400) may be removably secured to a trocar (330) using any other suitable components, features, or techniques.

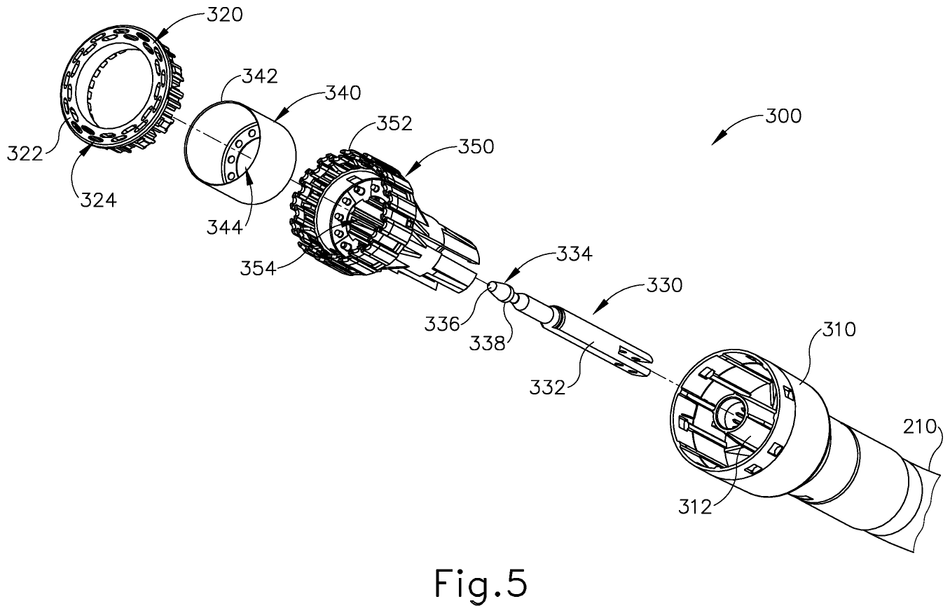

Stapling head assembly (300) is located at the distal end of shaft assembly (200). As shown in FIGS. 1 and 2, anvil (400) is configured to removably couple with shaft assembly (200), adjacent to stapling head assembly (300). As will be described in greater detail below, anvil (400) and stapling head assembly (300) are configured to cooperate to manipulate tissue in three ways, including clamping the tissue, cutting the tissue, and stapling the tissue. As best seen in FIGS. 4 and 5, stapling head assembly (300) of the present example comprises a tubular casing (310) housing a slidable staple driver member (350). A cylindraceous inner core member (312) extends distally within tubular casing (310). Tubular casing (310) is fixedly secured to an outer sheath (210) of shaft assembly (200), such that tubular casing (310) serves as a mechanical ground for stapling head assembly (300).

Trocar (330) is positioned coaxially within inner core member (312) of tubular casing (310). Trocar (330) is operable to translate distally and proximally relative to tubular casing (310) in response to rotation of a knob (130) located at the proximal end of handle assembly (100). Trocar (330) comprises a shaft (332) and a head (334). Head (334) includes a pointed tip (336) and an inwardly extending proximal surface (338). Head (334) and the distal portion of shaft (332) are configured for insertion in bore (422) of anvil (00). Proximal surface (338) is configured to complement features of latch members (430) to provide a snap fit between anvil (400) and trocar (330).

Staple driver member (350) is operable to actuate longitudinally within tubular casing (310) in response to activation of motor (160) as will be described in greater detail below. Staple driver member (350) includes two distally presented concentric annular arrays of staple drivers (352). Staple drivers (352) are arranged to correspond with the arrangement of staple forming pockets (414) described above. Thus, each staple driver (352) is configured to drive a corresponding staple into a corresponding staple forming pocket (414) when stapling head assembly (300) is actuated. Staple driver member (350) also defines a bore (354) that is configured to coaxially receive core member (312) of tubular casing (310).

A cylindraceous knife member (340) is coaxially positioned within staple driver member (350). Knife member (340) includes a distally presented, sharp circular cutting edge (342). Knife member (340) is sized such that knife member (340) defines an outer diameter that is smaller than the diameter defined by the inner annular array of staple drivers (352). Knife member (340) also defines an opening that is configured to coaxially receive core member (312) of tubular casing (310).

A deck member (320) is fixedly secured to tubular casing (310). Deck member (320) includes a distally presented deck surface (322) defining two concentric annular arrays of staple openings (324). Staple openings (324) are arranged to correspond with the arrangement of staple drivers (352) and staple forming pockets (414) described above. Thus, each staple opening (324) is configured to provide a path for a corresponding staple driver (352) to drive a corresponding staple through deck member (320) and into a corresponding staple forming pocket (414) when stapling head assembly (300) is actuated. It should be understood that the arrangement of staple openings (324) may be modified just like the arrangement of staple forming pockets (414) as described above. It should also be understood that various structures and techniques may be used to contain staples within stapling head assembly (300) before stapling head assembly (300) is actuated. Deck member (320) defines an inner diameter that is just slightly larger than the outer diameter defined by knife member (340). Deck member (320) is thus configured to allow knife member (340) to translate distally to a point where cutting edge (342) is distal to deck surface (322).

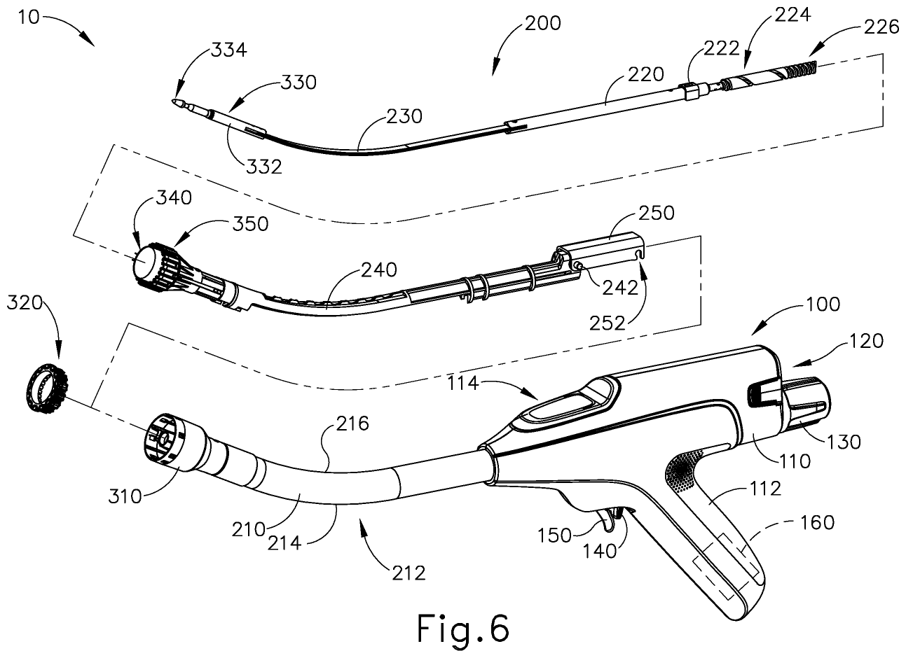

FIG. 6 shows various components of shaft assembly (200), which extends distally from handle assembly (100) and couples components of stapling head assembly (300) with components of handle assembly (100). In particular, and as noted above, shaft assembly (200) includes an outer sheath (210) that extends between handle assembly (100) and tubular casing (310). In the present example, outer sheath (210) is rigid and includes a preformed curved section (212) that is configured to facilitate positioning of stapling head assembly (300) within a patient's colon as described below. Curved section (212) includes an inner curve (216) and an outer curve (214).

Shaft assembly (200) further includes a trocar actuation rod (220) and a trocar actuation band assembly (230). The distal end of trocar actuation band assembly (230) is fixedly secured to the proximal end of trocar shaft (332). The proximal end of trocar actuation band assembly (230) is fixedly secured to the distal end of trocar actuation rod (220), such that trocar (330) will translate longitudinally relative to outer sheath (210) in response to translation of trocar actuation band assembly (230) and trocar actuation rod (220) relative to outer sheath (210). Trocar actuation band assembly (230) is configured to flex such that trocar actuation band assembly (230) may follow along the preformed curve in shaft assembly (200) as trocar actuation band assembly (230) is translated longitudinally relative to outer sheath (210). However, trocar actuation band assembly (230) has sufficient column strength and tensile strength to transfer distal and proximal forces from trocar actuation rod (220) to trocar shaft (332). Trocar actuation rod (220) is rigid. A clip (222) is fixedly secured to trocar actuation rod (220) and is configured to cooperate with complementary features within handle assembly (100) to prevent trocar actuation rod (220) from rotating within handle assembly (100) while still permitting trocar actuation rod (220) to translate longitudinally within handle assembly (100). Trocar actuation rod (220) further includes a coarse helical threading (224) and a fine helical threading (226).

Shaft assembly (200) further includes a stapling head assembly driver (240) that is slidably received within outer sheath (210). The distal end of stapling head assembly driver (240) is fixedly secured to the proximal end of staple driver member (350). The proximal end of stapling head assembly driver (240) is secured to a drive bracket (250) via a pin (242). It should therefore be understood that staple driver member (350) will translate longitudinally relative to outer sheath (210) in response to translation of stapling head assembly driver (240) and drive bracket (250) relative to outer sheath (210). Stapling head assembly driver (240) is configured to flex such that stapling head assembly driver (240) may follow along the preformed curve in shaft assembly (200) as stapling head assembly driver (240) is translated longitudinally relative to outer sheath (210). However, stapling head assembly driver (240) has sufficient column strength to transfer distal forces from drive bracket (250) to staple driver member (350).

### B. Exemplary User Input Features of Circular Stapling Instrument

As shown in FIG. 1, handle assembly (100) includes a pistol grip (112) and several components that are operable to actuate anvil (400) and stapling head assembly (300). In particular, handle assembly (100) includes knob (130), a safety trigger (140) a firing trigger (150), a motor (160), and a motor activation module (180). Knob (130) is coupled with trocar actuation rod (220) via a nut (not shown), such that coarse helical threading (224) will selectively engage a thread engagement feature within the interior of the nut; and such that fine helical threading (226) will selectively engage a thread engagement feature within the interior of knob (130). These complementary structures are configured such that trocar actuation rod (220) will first translate proximally at a relatively slow rate, then translate proximally at a relatively fast rate, in response to rotation of knob (130).

It should be understood that when anvil (400) is coupled with trocar (330), rotation of knob (130) will provide corresponding translation of anvil relative to stapling head assembly (300). It should also be understood that knob (130) may be rotated in a first angular direction (e.g., clockwise) to retract anvil (400) toward stapling head assembly (300); and in a second angular direction (e.g., counterclockwise) to advance anvil (400) away from stapling head assembly (300). Knob (130) may thus be used to adjust the gap distance between opposing surfaces (412, 322) of anvil (400) and stapling head assembly (300) until a suitable gap distance has been achieved.

In the present example, handle assembly (100) comprises a user feedback feature (114) that is configured to provide the operator with visual feedback indicating the positioning of anvil (400) in relation to stapling assembly (300). The operator may thus observe user feedback feature (114) while rotating knob (130), to confirm whether the suitable gap distance between anvil (400) and stapling assembly (300) has been achieved.

Firing trigger (150) is operable to activate motor (160) to thereby actuate stapling head assembly (300). Safety trigger (140) is operable to selectively block actuation of firing trigger (150) based on the longitudinal position of anvil (400) in relation to stapling head assembly (300). Handle assembly (100) also includes components that are operable to selectively lock out both triggers (140, 150) based on the position of anvil (400) relative to stapling head assembly (300). When triggers (140, 150) are locked out, firing trigger (150) is prevented from initiating actuation of stapling head assembly (300). Thus, trigger (150) is only operable to initiate actuation of stapling head assembly (300) when the position of anvil (400) relative to stapling head assembly (300) is within a predefined range.

In the present example, firing trigger (150) of the present example includes an integral actuation paddle. The paddle is configured to actuate a switch of motor activation module (180) (FIG. 1) when firing trigger (150) is pivoted to a fired position. Motor activation module (180) is in communication with control attachment (120) and motor (160), such that motor activation module (180) is configured to provide activation of motor (160) with electrical power from control attachment (120) in response to the paddle actuating the switch of motor activation module (180). Thus, motor (160) will be activated when firing trigger (150) is pivoted. This activation of motor (160) will actuate stapling head assembly (300) as described in greater detail below.

Control attachment (120) is operable to provide electrical power to a motor (160) as noted above. Control attachment (120) may be removably coupled with handle assembly (100) through a snap fit or in any other suitable fashion. It should be understood that control attachment (120) and handle assembly (100) may have complementary electrical contacts, pins and sockets, and/or other features that provide paths for electrical communication from control attachment (120) to electrically powered components in handle assembly (100) when control attachment (120) is coupled with handle assembly (100). As will be discussed in greater detail below, control attachment (120) can include an energy source (700) in electrical connection with a motor assembly (which can include motor (160) and transmission (612), as described below).

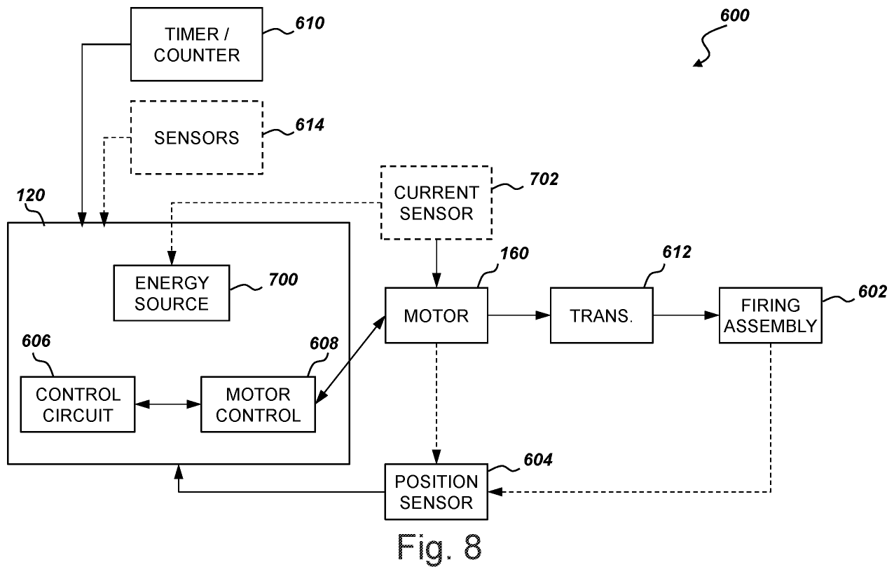

One aspect of the present disclosure is to provide a surgical stapler (10) with electronic functionality, such as functionalities to control motor (160) and other electrical components. Additional information about the electrical components that can be implemented with surgical stapler (10) is provided below with respect to FIG. 8. In general, surgical stapler (10) can include control circuit (606) that can include hardware and/or software to help control (i.e., generate or receive firing signals to and from motor (160) and/or firing assembly (602)) or to monitor the device (i.e., receive information from sensors (see discussion of FIG. 8)). Traditional staplers include a plurality of mechanical components that are contraindicated for repeated use. For example, knife members can wear, the device can be difficult to sterilize because of small crevasses therein, the stapler may not include reusable staple cartridges, etc. Accordingly, many staplers in the art are single-use devices. Single-use components can create excess waste. And in many scenarios, desirable features may not be employed on the staplers if they are intended to be disposable—i.e., it can be prohibitively expensive to create smart staplers that merely get discarded after use. To alleviate some of these burdens and to save costs, the present disclosure employs control attachment (120) that can be removable and reusable. The remainder of surgical stapler (10) (i.e., the components of the handle assembly (100), stapling head assembly (300), and anvil (400)) can be disposable. As such, the present surgical stapler (10) leverages the lifespan of electronic components (i.e., those in control attachment (120)) that do not face the mechanical wear present within the physical device, and the present surgical stapler (10) allows for more expensive, elaborate sensing modalities to be implemented within the stapler.

FIG. 2 provides illustration of such removable and reusable control attachment (120). Control circuit (606) can be positioned on or within control attachment (120). Control attachment (120) can further include energy source (700), which can include for example a disposable or rechargeable battery, including for example a lithium ion battery and the like. Control circuit (606), once control attachment (120) is connected with handle assembly (100), is in electrical communication with a motor assembly, which can include motor (160) and/or transmission (612) (see FIG. 8). Hereinafter reference to “motor assembly (160, 612)” will be understood to refer to one or more of motor (160) or transmission (612).

Handle assembly (100) can include a cavity (704) in which to receive control attachment (120). As described above, handle assembly (100) may include within cavity (704) electrical contacts, pins and sockets, and/or other features that provide paths for electrical communication from control attachment (120) to electrically powered components in handle assembly (100) when control attachment (120) is inserted into cavity (704). Hereinafter reference to “controller (606, 608)” will be understood to refer to one or more of control circuit (606) or motor controller (608). More information about motor controller (608) is provided with reference to FIG. 8 below. Controller (606, 608) can output a motor drive signal to the motor assembly (160, 612) such that the motor assembly (160, 612) drives at least a portion of the firing assembly (602) (see FIG. 8 and related discussion regarding firing assembly (602)). Controller (606, 608) can be in electrical communication with components of the handle assembly (100) via one or more hall effect switches and/or other types of magnetic switches. In some examples, controller (606, 608) can be in electrical communication with components of the handle assembly (100) via one or more inductive switches. As will be appreciated, magnetic switches and inductive switches can help to reduce fluid ingress into either handle assembly (100) or control attachment (120) while still maintaining a communicative connection between control attachment (120) and handle assembly (100). In some examples, controller (606, 608) can be in electrical communication with components of the handle assembly (100) via wireless communications, e.g., via near field communication (NFC) or other wireless technologies.

To further reduce fluid ingress and to also provide a sterile instrument, the control attachment (120) can be positioned within cavity (704), and once positioned as such, control attachment (120) can be aseptically sealed within cavity (704). In this example, mechanical components of a disposable surgical stapler (10) (e.g., handle assembly (100), stapling head assembly (300), and anvil (400)) can be pre-sterilized and packaged in a sterile packaging. Once opened and placed into the sterile environment for the medical procedure, a reusable control attachment (120) can be inserted into cavity (704) via an aseptic surgical procedure and then hermetically sealed so as to maintain sterility during a procedure. In some examples, therefore, handle assembly (100) can include a cover (706) configured to aseptically seal energy source (700) and control circuit (606) within cavity (704). Cover (706) can be attached to or integrated with control attachment (120), or in other examples, cover (706) can be attached to or integrated with handle assembly (100).

C. Exemplary Anastomosis Procedure with Circular Stapling Instrument

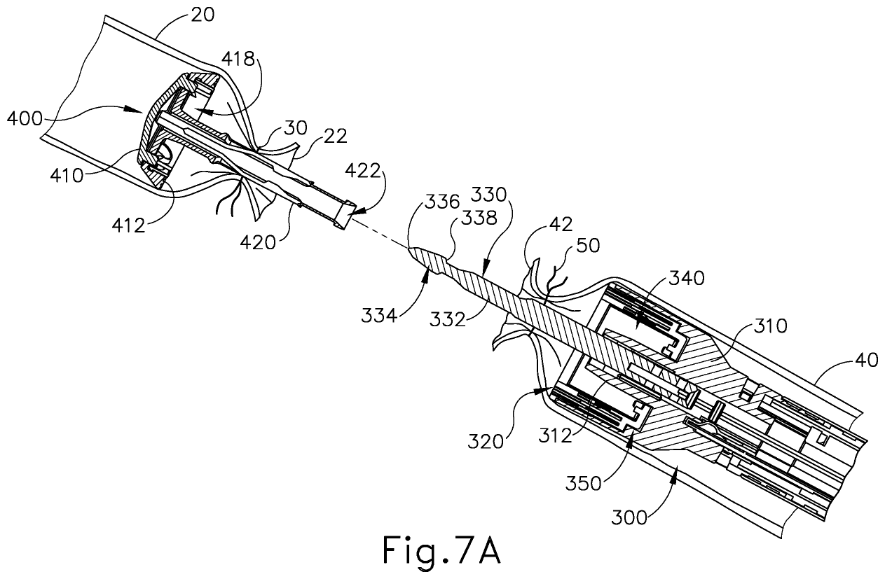

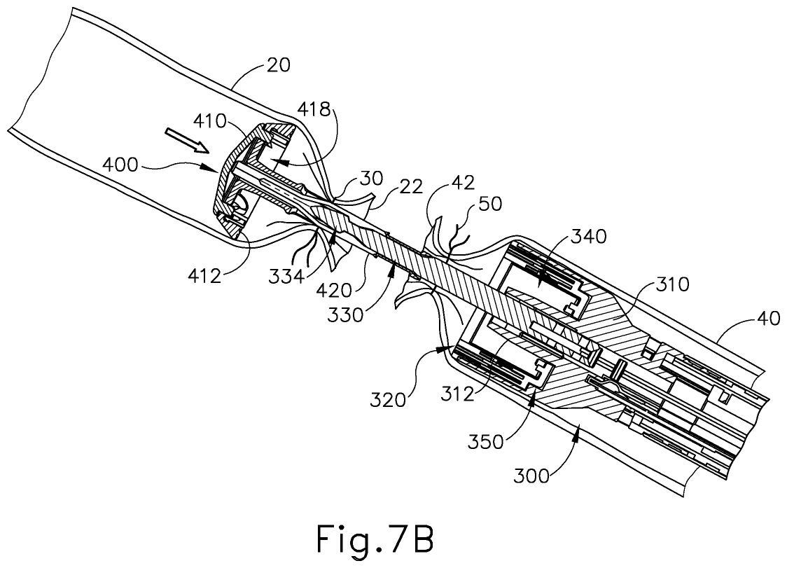

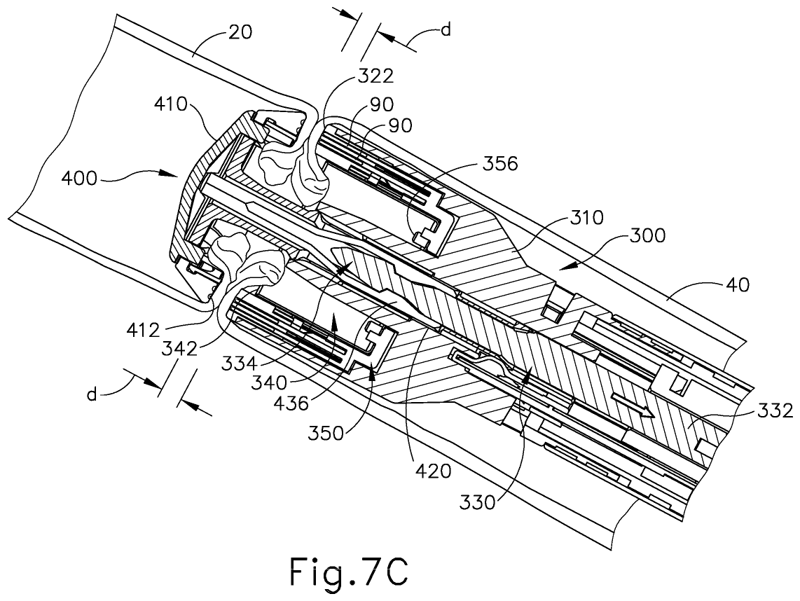

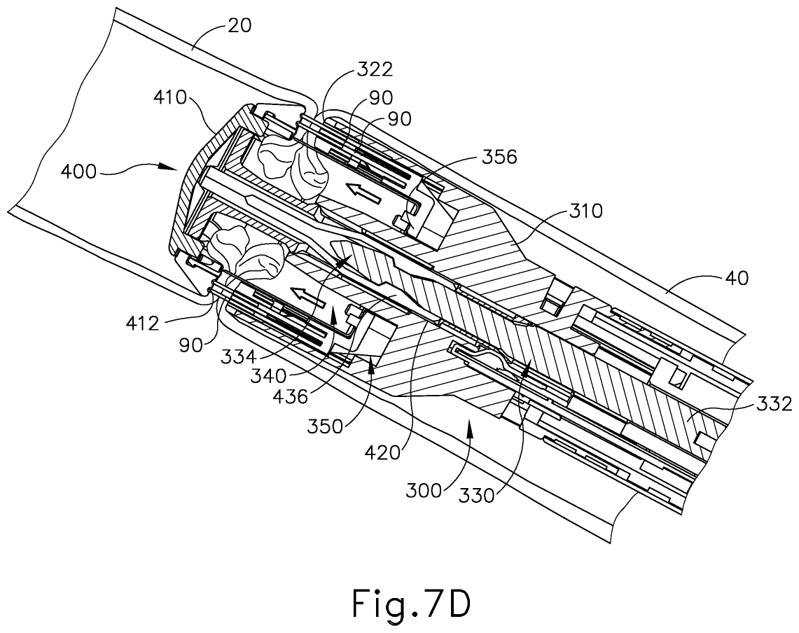

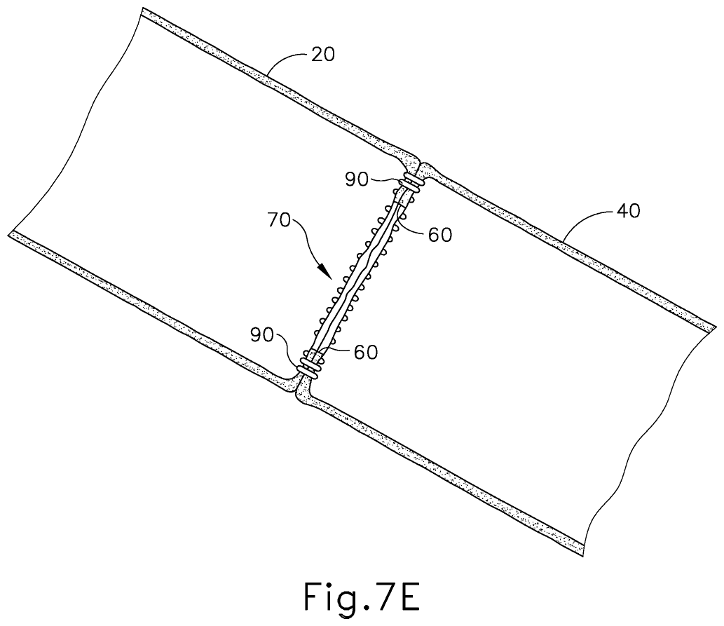

FIGS. 7A-7E show surgical stapler (10) being used to form an anastomosis (70) between two tubular anatomical structures (20, 40). By way of example only, the tubular anatomical structures (20, 40) may comprise sections of a patient's esophagus, sections of a patient's colon, other sections of the patient's digestive tract, or any other tubular anatomical structures. In some versions, one or more diseased portions of a patient's colon are removed, with the tubular anatomical structures (20, 40) of FIGS. 7A-7E representing the remaining severed portions of the colon.

As shown in FIG. 7A, anvil (400) is positioned in one tubular anatomical structure (20) and stapling head assembly (300) is positioned in another tubular anatomical structure (40). In versions where tubular anatomical structures (20, 40) comprise sections of a patient's colon, stapling head assembly (300) may be inserted via the patient's rectum. It should also be understood that the procedure depicted in FIGS. 7A-7E is an open surgical procedure, though the procedure may instead be performed laparoscopically. By way of example only, the surgical procedure may be performed laparoscopically. Various other suitable ways in which surgical stapler (10) may be used to form an anastomosis (70) in a laparoscopic procedure will be apparent to those of ordinary skill in the art in view of the teachings herein.

As shown in FIG. 7A, anvil (400) is positioned in tubular anatomical structure (20) such that shank (420) protrudes from the open severed end (22) of tubular anatomical structure (20). A purse-string suture (30) is provided about a mid-region of shank (420) to generally secure the position of anvil (400) in tubular anatomical structure (20). Similarly, stapling head assembly (300) is positioned in tubular anatomical structure (40) such that trocar (330) protrudes from the open severed end (42) of tubular anatomical structure (20). A purse-string suture (50) is provided about a mid-region of shaft (332) to generally secure the position of stapling head assembly (300) in tubular anatomical structure (40).

Next, anvil (400) is secured to trocar (330) by inserting trocar (330) into bore (422) as shown in FIG. 7B. Latch members (430) engage head (334) of trocar (330), thereby providing a secure fit between anvil (400) and trocar (330). The operator then rotates knob (130) while holding handle assembly (100) stationary via pistol grip (112). This rotation of knob (130) causes trocar (330) and anvil (400) to retract proximally, as described above. As shown in FIG. 7C, this proximal retraction of trocar (330) and anvil (400) compresses the tissue of tubular anatomical structures (20, 40) between surfaces (412, 322) of anvil (400) and stapling head assembly (300). The operator observes user feedback feature (114) to determine whether the gap distance (d) between opposing surfaces (412, 322) of anvil (400) and stapling head assembly (300) is appropriate; and makes any necessary adjustments via knob (130).

Once the operator has appropriately set the gap distance (d) via knob (130), the operator actuates safety trigger (140) to enable actuation of firing trigger (150). The operator then actuates firing trigger (150). This actuation of firing trigger (150) in turn actuates a switch of motor activation module (180), which in turn activates motor (160) to thereby actuate stapling head assembly (300) by driving knife member (340) and staple driver member (350) distally as shown in FIG. 7D. As knife member (340) translates distally, cutting edge (342) of knife member (340) cooperates with inner edge (416) of anvil (400), thereby shearing excess tissue that is positioned within annular recess (418) of anvil (400) and the interior of knife member (340).

As shown in FIG. 4, anvil (400) of the present example includes a breakable washer (417) within annular recess (418). This washer (417) is broken by knife member (340) when knife member (340) completes a full distal range of motion from the position shown in FIG. 7C to the position shown in FIG. 7D. The drive mechanism for knife member (340) may provide an increasing mechanical advantage as knife member (340) reaches the end of its distal movement, thereby providing greater force by which to break washer (417). Of course, breakable washer (417) may be omitted entirely in some versions. In versions where washer (417) is included, it should be understood that washer (417) may also serve as a cutting board for knife member (340) to assist in cutting of tissue. Such a cutting technique may be employed in addition to or in lieu of the above-noted shearing action between inner edge (416) and cutting edge (342).

As staple driver member (350) translates distally from the position shown in FIG. 7C to the position shown in FIG. 7D, staple driver member (350) drives staples (90) through the tissue of tubular anatomical structures (20, 40) and into staple forming pockets (414) of anvil (400). Staple forming pockets (414) deform the driven staples (90) into a “B” shape as is known in the art. The formed staples (90) thus secure the ends of tissue together, thereby coupling tubular anatomical structure (20) with tubular anatomical structure (40).

After the operator has actuated stapling head assembly (300) as shown in FIG. 7D, the operator rotates knob (130) to drive anvil (400) distally away from stapling head assembly (300), increasing the gap distance (d) to facilitate release of the tissue between surfaces (412, 322). The operator then removes surgical stapler (10) from the patient, with anvil (400) still secured to trocar (330). Referring back to the example where the tubular anatomical structures (20, 40) comprise sections of a patient's colon, surgical stapler (10) may be removed via the patient's rectum. With surgical stapler (10) removed, the tubular anatomical structures (20, 40) are left secured together by two annular arrays of staples (90) at an anastomosis (70) as shown in FIG. 7E. The inner diameter of the anastomosis (70) is defined by the severed edge (60) left by knife member (340).

In some instances, it may be desirable to change the configuration and arrangement of staple forming pockets (414) in anvil (400). It should be understood that reconfiguring and rearranging staple forming pockets (414) may result in reconfiguration and rearrangement of staples (90) that are formed by staple forming pockets (414). For instance, the configuration and arrangement of staple forming pockets (414) may affect the structural integrity of an anastomosis (70) that is secured by staples (90). In addition, the configuration and arrangement of staple forming pockets (414) may affect the hemostasis that is achieved at an anastomosis (70) that is secured by staples (90). The following description relates to several exemplary variations of anvil (400), providing staple forming pocket configurations and arrangements that differ from those of staple forming pockets (414).

It should be understood that the various alternatives to anvil (400) described below may be readily used with surgical stapler (10), in place of anvil (400). It should also be understood that, in some instances, the configuration and arrangement of staple openings (324) in deck member (320) may need to be varied in order to complement the configuration and arrangement of the alternative staple forming pockets described below. Various suitable ways in which the alternatives to anvil (400) described below may be incorporated into surgical stapler (10) will be apparent to those of ordinary skill in the art in view of the teachings herein. Additional information about circular staplers can be found in U.S. Publication No. 2018/0132849, entitled Staple Forming Pocket Configurations For Circular Surgical Stapler Anvil, which is incorporated herein by reference in its entirety.

FIG. 8 is block diagram of an example firing system (600) for a surgical stapler (10) such as the surgical circular stapling instrument (10) illustrated in FIG. 1, variations thereof, or an alternative thereto as understood by a person skilled in the pertinent art. The firing system (600) is configured to control longitudinal translation of a firing assembly (602) of the surgical circular stapling instrument (10). The firing assembly (602) can include the slidable staple driver member (350), deck member (320), and knife member (340) as illustrated in FIG. 5, alternatives thereto, variations thereof, and/or sub combinations thereof. The firing assembly (602) is configured to deploy staples and may also cut tissue during a firing stroke of the surgical instrument (10). The firing system (600) can include transmission (612) configured to convert the rotational movement of a rotor of the motor (160) into longitudinal movement of the firing assembly (602). As described above, motor (160) and transmission (612) are collectively referred to herein as a motor assembly (160, 612) (but the motor assembly throughout this disclosure could also mean one of the motor (160) or the transmission (612)). Note that the motor (160) as illustrated, may represent more than one motor. As described above, surgical stapler (10) can include motor activation module (180) and firing trigger (150). Activation of firing trigger (150) actuates a switch of motor activation module (180) to activate motor assembly (160, 612). Controller (606, 608) can transmit the aforementioned motor drive signal in response to activation of motor assembly (160, 612) by motor activation module (180).

The position, movement, displacement, and/or translation of one or more components of the firing assembly (602), can be measured by one or more position sensors (604). The position sensor(s) (604) may be configured to detect movement of the firing assembly (602) and/or rotation of the rotor of the motor (160). The position sensor(s) (604) can otherwise be configured to sense a physical parameter of the surgical circular stapling instrument (10) and provide an electrical signal output indicative of the knife member (340), slidable staple driver member (350), or another portion of the firing assembly (602) which translates longitudinally through the stapling instrument (10) during a firing stroke. Additionally, or alternatively, the position sensor (604) can be configured to detect which staples (90) have been deployed and which have not been deployed. Deployment status of staples (90) may provide an indication of a position of the distal portion of the firing assembly (602).

The position sensor(s) (604) may be located in the stapling head assembly (300) and/or at any other portion of the surgical circular stapling instrument (10). In some embodiments, the position sensor(s) (604) include an encoder configured to provide a series of pulses to the control attachment (120) as the rotor of the motor (160) rotates and the firing assembly (602) is translated longitudinally. The control attachment (120) may track the pulses to determine the position of a component of the firing assembly (602). Other suitable position sensors may be used, including, for example, a proximity sensor. Other types of position sensors may provide other signals indicating motion of a component of the firing assembly (602). In some embodiments, the position sensor(s) (604) may be omitted. For instance, where the motor (160) is a stepper motor, the control attachment (120) may track the position of a component of the firing assembly (602) by aggregating the number and direction of steps that the motor (160) has been instructed to execute.

The control attachment (120) is illustrated as including a control circuit (606) and motor controller (608), which are illustrated as two separate blocks. The control circuit (606) and motor controller (608) and may be separate circuits or may be integrated as a single circuit. The control circuit (606) is configured to provide a motor setpoint signal output to the motor controller (608). The motor setpoint signal is indicative of a target speed of the firing assembly (602). The motor controller (608) is configured to provide a motor drive signal to the motor (160) such that the motor drive signal is based on the motor setpoint signal and intended to drive the motor (160) so that the firing assembly (602) is driven to the target speed.

Note that, during a firing stroke, the actual speed of the firing assembly (602) may not precisely match the target speed. The motor controller (608) is configured to drive the firing assembly (602) to the target speed, meaning, as the actual speed of the firing assembly (602) deviates from the target speed, the motor controller (608) is configured to adjust the speed of the firing assembly (602) so that the speed of the firing assembly more closely matches the target speed.

The control circuit (606) and the motor controller (608) may include one or more processors and memory (i.e., one or more non-transitory computer-readable medium) with instructions that can be executed by the one or more processors to cause the control circuit (606) and the motor controller (608) to drive the motor (160). The control circuit (606) and/or motor controller (608) can include a feedback controller, which can be one of any feedback controllers, including, but not limited to a PID, a State Feedback, LQR, and/or an Adaptive controller, for example. The control circuit (606) and/or motor controller (608) can include a power source to convert the signal from the feedback controller into a physical input such as a constant voltage, pulse width modulated (PWM) voltage, frequency modulated voltage, current, torque, and/or force, for example.

The firing system (600) can include a timer/counter circuit (610) configured to provide an output signal, such as elapsed time or a digital count, to the control circuit (606). The control circuit (606) is configured to determine a position of the firing assembly (602) based on the signal from the position sensor(s) (604) and correlate the position of the firing assembly (602) with the output of the timer/counter circuit (610) such that the control circuit (606) can determine the position of one or more components of the firing assembly (602) at a specific time relative to a starting position. The timer/counter circuit (610) may be configured to measure elapsed time, count external events, or time external events.

At the beginning of a firing stroke the control circuit (606) can be configured to provide a motor set point signal to the motor control (608) that indicates a fixed initial speed. The motor controller (608) can be configured to provide a motor drive input signal to the motor (160) that adjusts power drawn by the motor (160) so that the motor (160) is driven approximately at the fixed initial speed. In some embodiments, the fixed initial speed is approximately 12 mm/s to approximately 16 mm/s, and more preferably at approximately 12 mm/s.

In some embodiments, the control circuit (606) can be configured to initiate a pause in response to detecting the speed error. The control circuit (606) is configured to set the target speed to zero for a pause time duration in response to detecting the speed error. The motor setpoint signal output from the control circuit (606) to the motor controller (608) indicates a target speed of zero during the pause time duration. During a pause, tissue is compressed between the anvil (400) and deck member (320) of the surgical circular stapling instrument (10). During at least a portion of the pause, the firing assembly (602) can remain stationary at a paused position.

The firing system (600) may optionally include a current sensor (702) configured to measure the current drawn by the motor (160) from the energy source (700). In some embodiments the control circuit (606) is configured to detect the speed error when the actual speed of the firing assembly (602) is less than the target speed by the speed threshold and electrical current driving the motor (160) is greater than a current threshold.

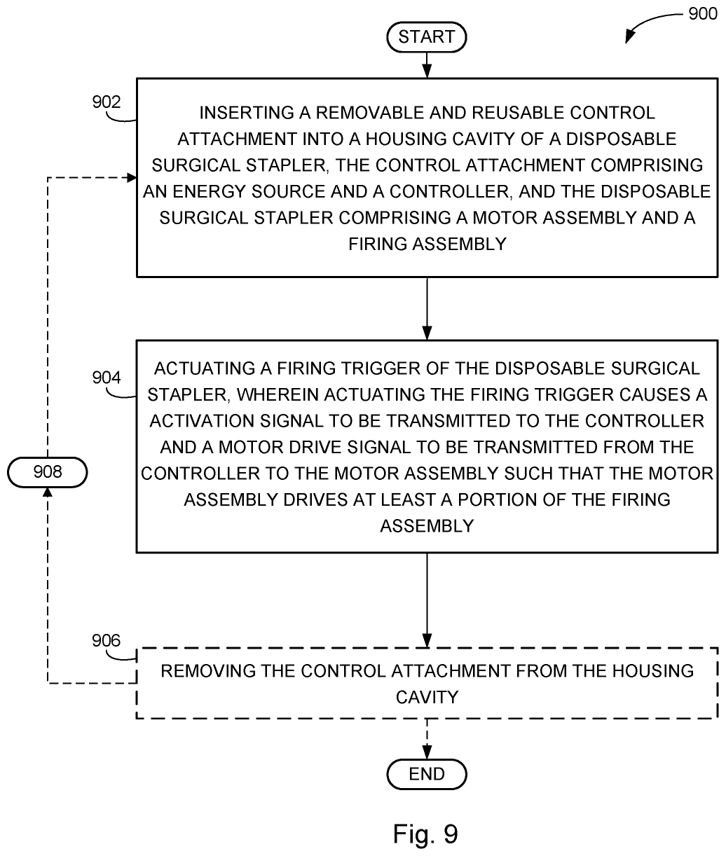

FIG. 9 is a flowchart depicting an example method (900) of operating a surgical stapler (10), according to one aspect of the present disclosure. Method (900) includes inserting (902) a removable and reusable control attachment (120) into a first housing cavity (704) of a first disposable surgical stapler (10). The control attachment (120) includes an energy source (700) and a controller (606, 608). The first disposable surgical stapler (10) comprising a first motor assembly (160, 612) and a first firing assembly (602). Method (900) includes actuating (904) a first firing trigger (150) of the first disposable surgical stapler (10). Actuating the first firing trigger (150) causes a first activation signal to be transmitted to the controller (606, 608) and a first motor drive signal to be transmitted from the controller (606, 608) to the first motor assembly (160, 612) such that the first motor assembly (160, 612) drives at least a portion of the first firing assembly (602). Method (900) includes removing (906) the control attachment (120) from the first housing cavity (704). Method (900) can end after the removing (906) step. In other example, method (900) can be repeated by inserting (908) the removable and reusable control attachment (120) into a second housing cavity (704) of a second disposable surgical stapler (10). Steps (904) and (906) can be repeated for the second disposable surgical stapler (10).

The technology described herein can further be implemented by any of the following numbered clauses:

Clause 1: A stapler system comprising: (A) a disposable surgical stapler (10) comprising: a firing assembly (602) configured to deploy staples (90) from a stapling head assembly (300); a handle assembly (100) mechanically coupled to the firing assembly (602); a motor assembly (160, 612) disposed within the handle assembly and configured to drive the firing assembly (602); and (B) a removable and reusable control attachment (120) comprising: an energy source (700) in electrical connection with the motor assembly (160, 612); and a controller (606, 608) in electrical communication with the motor assembly (160, 612) and configured to output a motor drive signal to the motor assembly (160, 612) such that the motor assembly (160, 612) drives at least a portion of the firing assembly (602).

Clause 2: The stapler system of Clause 1, wherein the handle assembly (100) comprises a housing cavity (704), wherein the control attachment (120) is positionable within the housing cavity (704), and wherein once the control attachment (120) is positioned within the housing cavity (704), the control attachment (120) is aseptically sealed within the housing cavity (704).

Clause 3: The stapler system of Clause 2, wherein the handle assembly (100) comprises a cover (704) configured to aseptically seal the energy source (700) and the controller (606, 608) within the housing cavity (704).

Clause 4: The stapler system of any of the preceding Clauses, wherein the controller (606, 608) is in electrical communication with the motor assembly (160, 612) via a hall effect switch.

Clause 5: The stapler system of any of Clauses 1 to 3, wherein the controller (606, 608) is in electrical communication with the motor assembly (160, 612) via an inductive switch.

Clause 6: The stapler system of any of the preceding Clauses, wherein the disposable surgical stapler (10) further comprises: a motor activation module (180); and a firing trigger (150), wherein activation of the firing trigger (150) actuates a switch of motor activation module (180) to activate the motor assembly (160, 612), and wherein the controller (606, 608) transmits the motor drive signal in response to activation of the motor assembly (160, 612) by the motor activation module (180).

Clause 7: The stapler system of any of the preceding Clauses, wherein the disposable surgical stapler (10) further comprises a current sensor (702) configured to measure the current drawn by the motor assembly (160, 612) from the energy source (700).

Clause 8: The stapler system of any of Clauses 1 to 6, wherein the control attachment (120) further comprises a current sensor (702) configured to measure the current drawn by the motor assembly (160, 612) from the energy source (700).

Clause 9: The stapler system of any of the preceding Clauses, wherein the energy source (700) is a rechargeable battery.

Clause 10: A method of operating a surgical stapler (10) comprising: inserting a removable and reusable control attachment (120) into a first housing cavity (704) of a first disposable surgical stapler (10), the control attachment (120) comprising an energy source (700) and a controller (606, 608), and the first disposable surgical stapler (10) comprising a first motor assembly (160, 612) and a first firing assembly (602); actuating a first firing trigger (150) of the first disposable surgical stapler (10), wherein actuating the first firing trigger (150) causes a first activation signal to be transmitted to the controller (606, 608) and a first motor drive signal to be transmitted from the controller (606, 608) to the first motor assembly (160, 612) such that the first motor assembly (160, 612) drives at least a portion of the first firing assembly (602); and removing the control attachment (120) from the first housing cavity (704).

Clause 11: The method of Clause 10 further comprising recharging the energy source (700).

Clause 12: The method of Clause 11 further comprising: inserting the control attachment (120) into a second housing cavity (704) of a second disposable surgical stapler (10), the second disposable surgical stapler (10) comprising a second motor assembly (160, 612) and a second firing assembly (602); actuating a second firing trigger (150) of the second disposable surgical stapler (10), wherein actuating the second firing trigger (150) causes a second activation signal to be transmitted to the controller (606, 608) and a second motor drive signal to be transmitted from the controller (606, 608) to the second motor assembly (160, 612) such that the second motor assembly (160, 612) drives at least a portion of the second firing assembly (602) and drive a plurality of staples; and removing control attachment (120) from the second housing cavity (704).

Clause 13: The method of any of Clauses 10 to 12, wherein once the control attachment (120) is positioned within the first housing cavity (704), the control attachment (120) is aseptically sealed within the first housing cavity (704).

Clause 14: The method of any of Clauses 10 to 13 further comprising closing a cover (704) on the first disposable surgical stapler (10) to aseptically seal the control attachment (120) with the first housing cavity (704).

Clause 15: The method of any of Clauses 10 to 14, wherein the controller (606, 608) is in electrical communication with the motor assembly (160, 612) via a hall effect switch or via an inductive switch.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometric s, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

The invention is not necessarily limited to the examples described, which can be varied in construction and detail. The terms “distal” and “proximal” are used throughout the preceding description and are meant to refer to a positions and directions relative to the physician or user holding surgical stapler 10. As such, “distal” or distally” refer to a position distant to or a direction away from the person gripping surgical stapler 10. Similarly, “proximal” or “proximally” refer to a position near or a direction towards the person grasping pistol grip 112 (i.e., toward an operator of surgical stapler 10). Furthermore, the singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g., “about 90%” may refer to the range of values from 80.001% to 99.999%.

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

1. A stapler system comprising:
(A) a disposable surgical stapler comprising:
a firing assembly configured to deploy staples from a stapling head assembly;
a handle assembly mechanically coupled to the firing assembly;
a motor assembly disposed within the handle assembly and configured to drive the firing assembly; and
(B) a removable and reusable control attachment comprising:
an energy source in electrical connection with the motor assembly; and
a controller in electrical communication with the motor assembly and configured to output a motor drive signal to the motor assembly such that the motor assembly drives at least a portion of the firing assembly.

2. The stapler system of claim 1, wherein the handle assembly comprises a housing cavity, wherein the control attachment is positionable within the housing cavity, and wherein once the control attachment is positioned within the housing cavity, the control attachment is aseptically sealed within the housing cavity.

3. The stapler system of claim 2, wherein the handle assembly comprises a cover configured to aseptically seal the energy source and the controller within the housing cavity.

4. The stapler system of claim 1, wherein the controller is in electrical communication with the motor assembly via a hall effect switch.

5. The stapler system of claim 1, wherein the controller is in electrical communication with the motor assembly via an inductive switch.

6. The stapler system of claim 1, wherein the disposable surgical stapler further comprises:
a motor activation module; and
a firing trigger,
wherein activation of the firing trigger actuates a switch of motor activation module to activate the motor assembly, and
wherein the controller transmits the motor drive signal in response to activation of the motor assembly by the motor activation module.

7. The stapler system of claim 1, wherein the disposable surgical stapler further comprises a current sensor configured to measure the current drawn by the motor assembly from the energy source.

8. The stapler system of claim 1, wherein the control attachment further comprises a current sensor configured to measure the current drawn by the motor assembly from the energy source.

9. The stapler system of claim 1, wherein the energy source is a rechargeable battery.

10. A method of operating a surgical stapler comprising:
inserting a removable and reusable control attachment into a first housing cavity of a first disposable surgical stapler, the control attachment comprising an energy source and a controller, and the first disposable surgical stapler comprising a first motor assembly and a first firing assembly;
actuating a first firing trigger of the first disposable surgical stapler, wherein actuating the first firing trigger causes a first activation signal to be transmitted to the controller and a first motor drive signal to be transmitted from the controller to the first motor assembly such that the first motor assembly drives at least a portion of the first firing assembly; and
removing the control attachment from the first housing cavity.

11. The method of claim 10 further comprising recharging the energy source.

12. The method of claim 11 further comprising:
inserting the control attachment into a second housing cavity of a second disposable surgical stapler, the second disposable surgical stapler comprising a second motor assembly and a second firing assembly;
actuating a second firing trigger of the second disposable surgical stapler, wherein actuating the second firing trigger causes a second activation signal to be transmitted to the controller and a second motor drive signal to be transmitted from the controller to the second motor assembly such that the second motor assembly drives at least a portion of the second firing assembly and drive a plurality of staples; and
removing control attachment from the second housing cavity.

13. The method of claim 10, wherein once the control attachment is positioned within the first housing cavity, the control attachment is aseptically sealed within the first housing cavity.

14. The method of claim 10 further comprising closing a cover on the first disposable surgical stapler to aseptically seal the control attachment with the first housing cavity.

15. The method of claim 10, wherein the controller is in electrical communication with the motor assembly via a hall effect switch or via an inductive switch.
